(19) United States
(12) Patent Application Publication
(10) Pub. No.: US 2024/0057848 A1
(43) Pub. Date: Feb. 22, 2024

(54) CONTROL DEVICE AND CONTROL METHOD

(71) Applicant: OLYMPUS CORPORATION, Tokyo (JP)

(72) Inventors: Naoki FUKATSU, Tokyo (JP); Hiromasa FUJITA, Tokyo (JP)

(73) Assignee: OLYMPUS CORPORATION, Tokyo (JP)

(21) Appl. No.: 18/385,564

(22) Filed: Oct. 31, 2023

Related U.S. Application Data
(63) Continuation of application No. PCT/JP2021/017474, filed on May 7, 2021.

(51) Int. Cl. A61B 1/00; A61B 1/015

(52) U.S. Cl. CPC A61B 1/000094; A61B 1/015

(57) ABSTRACT

A control device includes a processor. The processor acquires an endoscope image that is an image captured by an endoscope; determines whether suction is needed, based on the endoscope image; when the suction is determined to be needed, implements control to carry out the suction; determines a state of a subject based on a change in the endoscope image when the suction is performed; and performs control regarding suction based on the state of the subject.

### CROSS REFERENCE TO RELATED APPLICATIONS

This application is a continuation of International Patent Application No. PCT/JP2021/017474, having an international filing date of May 7, 2021, which designated the United States, the entirety of which is incorporated herein by reference.

### BACKGROUND OF THE INVENTION

Japanese Unexamined Patent Application Publication No. H05-245100 discloses an internal celomic pressure adjusting device for an endoscope for controlling internal celomic pressure at a constant level. This internal celomic pressure adjusting device includes a suction tube which is made to detachably communicate with a forceps channel of an endoscope, a suction means connected and communicated with the suction tube, a pressure detection means for detecting a pressure in the suction tube, and a suction control means which operates by an output signal from the pressure detection means to control the action of the suction means.

### SUMMARY OF THE INVENTION

In accordance with one of some aspect, there is provided a control device, including:
a processor, wherein
the processor is configured to:
acquire an endoscope image that is an image captured by an endoscope;
determine whether suction is needed, based on the endoscope image;
when the suction is determined to be needed, implement control to carry out the suction;
determine a state of a subject based on a change in the endoscope image when the suction is performed; and
perform control regarding suction based on the state of the subject.

In accordance with one of some aspect, there is provided a control method, comprising:
acquiring an endoscope image that is an image captured by an endoscope;
determining whether suction is needed, based on the endoscope image;
when the suction is determined to be needed, implementing control to carry out the suction;
determining a state of a subject based on a change in the endoscope image when the suction is performed; and
performing control regarding suction based on the state of the subject.

### BRIEF DESCRIPTION OF THE DRAWINGS

FIG. 1 illustrates a configuration example of a control device.

FIG. 2 is an explanatory view of parts of the large intestine.

FIG. 3 illustrates a first detailed configuration example of the control device.

FIG. 4 is a flowchart showing an example of automatic suction control when performing shaft retention and shortening method in the sigmoid colon.

FIG. 5 illustrates a first example of suction strength determination.

FIG. 6 is a flowchart showing an example of automatic suction control based on inspection status other than distance.

FIG. 7 illustrates a second example of suction strength determination.

FIG. 8 illustrates a third detailed configuration example of the control device.

FIG. 9 is a flowchart showing an example of automatic air supply control.

FIG. 10 illustrates detailed configuration examples of an endoscope, a suction device, and an air supply device.

FIG. 11 illustrates a fourth detailed configuration example of the control device.

FIG. 12 is a flowchart showing an example of luminal direction presentation.

FIG. 13 illustrates an example of a luminal direction presentation screen.

FIG. 14 illustrates a configuration example of an endoscope system.

FIG. 15 illustrates configuration examples of an endoscope and an endoscope shape acquisition sensor.

FIG. 16 illustrates a fifth detailed configuration example of the control device.

### DETAILED DESCRIPTION

The following disclosure provides many different embodiments, or examples, for implementing different features of the provided subject matter. These are, of course, merely examples and are not intended to be limiting. In addition, the disclosure may repeat reference numerals and/or letters in the various examples. This repetition is for the purpose of simplicity and clarity and does not in itself dictate a relationship between the various embodiments and/or configurations discussed. Further, when a first element is described as being “connected” or “coupled” to a second element, such description includes embodiments in which the first and second elements are directly connected or coupled to each other, and also includes embodiments in which the first and second elements are indirectly connected or coupled to each other with one or more other intervening elements in between.

### 1. Configuration Example

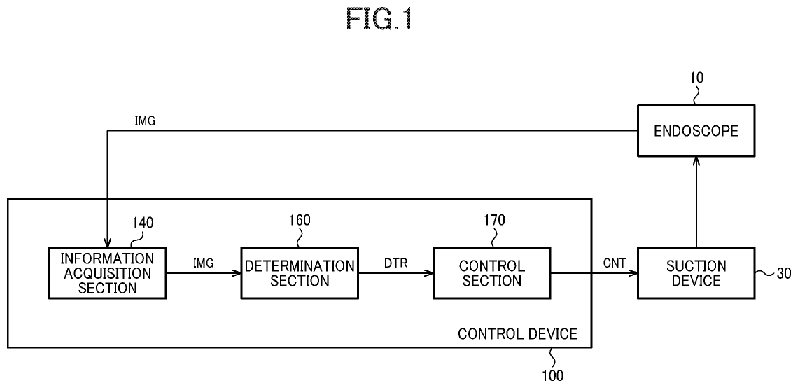

FIG. 1 illustrates a configuration example of a control device 100. The control device 100 includes an information acquisition section 140, a determination section 160, and a control section 170. In the following, a basic configuration example of a control device that performs suction control is described. The detailed configuration example of the control device, and the correspondence between the detailed configuration example and the basic configuration example is described later.

The information acquisition section 140 acquires an endoscope image IMG, which is an image captured by an endoscope 10. The endoscope 10, which is also called a scope, is inserted into the patient's body to capture images inside the patient's body. Specifically, the endoscope image IMG refers to images of individual frames of a moving image captured by the endoscope 10. The insertion of the endoscope 10 into the body may be performed by a doctor, or automatically or semi-automatically performed by an automatic insertion/removal device.

The determination section 160 performs determination as to whether or not the suction is needed based on the endoscope image IMG. Specifically, the determination section 160 performs an image recognition process on the endoscope image IMG to determine whether the current situation requires suction or does not require suction, and based on the recognition result, outputs a determination signal DTR indicating whether or not suction is needed. For example, the determination section 160 may determine an insertion status by an image recognition process, and then perform determination as to whether or not suction is needed based on the insertion status. Alternatively, the determination section 160 may obtain the determination signal DTR directly from the endoscope image IMG by an image recognition process. Although the determination as to whether or not suction is needed is described here, the determination section 160 may further determine the suction strength, as described later.

If the determination section 160 determines that suction is needed, the control section 170 implements control to carry out the suction. Specifically, when the determination signal DTR indicating that suction is needed is input, the control section 170 outputs a control signal CNT that turns on the suction to the suction device 30, and, when the determination signal DTR indicating that suction is not needed is input, the control section 170 outputs a control signal CNT that turns off the suction to the suction device 30. The endoscope 10 has a suction tube for sucking gas, liquid, or tissue fragments from the body cavity. The suction device 30 is connected to the suction tube, and performs suction when the suction ON control signal CNT is input, and does not perform suction when the suction OFF control signal CNT is input.

During the suctioning upon endoscope insertion, there is a demand to perform appropriate suction depending on the situation. The suction control to control only the internal celomic pressure at a constant level, as in the above-mentioned Japanese Unexamined Patent Application Publication No. H05-245100, may not be able to achieve appropriate suction according to the situation. For example, if the lumen is excessively opened at the time of insertion, suction is preferably automatically performed. Also, when the distal end of the endoscope comes closer to luminal inner wall due to suction upon the insertion, the suction is preferably turned off or weakened. Also, in the case of red ball state, where the image appears reddish because of the excessively short distance between the distal end of the endoscope and the mucosa, the suction is preferably turned off. These suction controls are difficult to achieve only by the control of making the internal pressure constant.

According to the present embodiment, since the determination as to whether or not the suction is needed is performed based on the endoscope image, it is possible to appropriately perform automatic control of ON and OFF of the suction according to the situation recognized from the endoscope image. This enables suction control, which has been difficult to achieve only by the control of making the internal pressure constant. The present embodiment is applicable to a variety of situations; however, as an example, suction can be performed if a situation where the lumen is excessively opened during the insertion is recognized from the endoscope image. Also, suction can be turned off if a situation where the distal end of the endoscope comes closer to luminal inner wall due to suction upon the insertion, or a red ball state where the image appears reddish because of the excessively short distance between the distal end of the endoscope and the mucosa is recognized from the endoscope image.

The determination section 160 determines whether or not the suction is needed according to the distance between the mucosa of the subject and the distal end section of the endoscope 10 based on the endoscope image IMG. Specifically, the determination section 160 performs determination as to whether or not the distance between the mucosa of the subject and the distal end section of the endoscope 10 is longer than a predetermined distance based on the endoscope image IMG. If it is determined that the distance is longer than the predetermined distance, it is determined that suction is needed. However, the determination section 160 may obtain the determination result as to whether or not the suction is needed directly from the endoscope image IMG by performing an image recognition process with respect to the endoscope image IMG in which the determination result as to whether or not the suction is needed changes depending on the distance, without obtaining the distance itself. The predetermined distance is, for example, the optimal distance with which the folds of the large intestine can be flipped by an angle operation and a torque operation of the endoscope 10 in the shaft retention and shortening method.

According to the present embodiment, suction can be automatically and appropriately controlled according to the distance between the mucosa of the subject and the distal end section of the endoscope 10, which is recognized from the endoscope image IMG. In cases such as the shaft retention and shortening method in colonoscopy, where the operation is performed while maintaining an appropriate distance between the mucosa of the subject and the distal end section of the endoscope 10, automatic control can be performed for the suction to maintain such an appropriate distance.

The determination section 160 determines whether or not the suction is needed based on the change in the endoscope image IMG when the suction is performed. The “change in endoscope image IMG when the suction is performed” refers to the change in endoscope image IMG over time before and after the suction, during the suction, or both. Specifically, the determination section 160 recognizes what kind of change have occurred in the endoscope image IMG through an image recognition process with respect to the endoscope image IMG, and determines whether or not the suction is needed according to the content of the recognized change. However, the determination section 160 may obtain the determination result as to whether or not the suction is needed directly from the endoscope image IMG by performing an image recognition process with respect to the endoscope image IMG in which the determination result as to whether or not the suction is needed changes depending on what kind of change has occurred in the endoscope image IMG, without recognizing the change itself.

According to the present embodiment, suction can be automatically and appropriately controlled according to changes in the endoscope image IMG when suction is performed. As an example, in the case where operation is performed while maintaining an appropriate distance between the mucosa of the subject and the distal end section of the endoscope 10 as in the shaft retention and shortening method in colonoscopy, the suction can be automatically turned off when the appropriate distance cannot be ensured even after the suction or when adhesions or other problems are found.

For example, if the determination section 160 determines that the distance between the mucosa of the subject and the distal end section of the endoscope 10 is not shorter than a predetermined distance based on the change in the endoscope image IMG when suction is performed, the determination section 160 determines not to perform the suction. As a result, the suction can be turned off when the optimal distance cannot be ensured by the suction for some reason.

Further, as another example, the determination section 160 determines the presence or absence of adhesion in the subject based on the change in the endoscope image IMG when suction is performed, and if it is determined that adhesion is present, the determination section 160 determines not to perform the suction. As a result, the suction can be turned off when the optimal distance cannot be ensured due to the adhesion.

Further, as still another example, the determination section 160 determines an operation plan that shows the operation procedures of the endoscope 10 based on the change in the endoscope image IMG when suction is performed, and determines whether or not the suction is needed according to the determined operation plan. This makes it possible to switch between a plurality of operation plans including the shaft retention and shortening method, the shaft retention and shortening method in consideration of adhesion, etc., the loop method, and the like, and perform appropriate automatic suction control in each operation plan.

The determination section 160 determines whether or not the suction is needed according to the position of the endoscope 10 in the lumen based on the endoscope image IMG. This position of the endoscope 10 refers to the position of the distal end section of the endoscope 10. Specifically, in the case where the lumen is divided into a plurality of sites, the position of the endoscope 10 refers to the site where the distal end section of the endoscope 10 is located, or it may refer to a more detailed position within the site. The determination section 160 determines the site where the distal end section of the endoscope 10 is located by an image recognition process with respect to the endoscope image IMG, and performs determination as to whether or not the suction is needed according to the site. However, the determination section 160 may obtain the determination result of whether or not the suction is needed directly from the endoscope image IMG by performing an image recognition process with respect to the endoscope image IMG in which the determination result as to whether or not the suction is needed changes depending on the site where the distal end section of the endoscope 10 is located, without determining the site itself.

According to the present embodiment, suction can be automatically and appropriately controlled according to the position of the endoscope 10 in the lumen. As an example, when the endoscope 10 makes its way through the sigmoid colon according to the shaft retention and shortening method in colonoscopy, when the endoscope 10 is located in the sigmoid colon, an appropriate distance with the distal end section of the endoscope 10 can be maintained by automatic suction control.

For example, the determination section 160 determines that suction is needed when it is determined that the endoscope 10 is located in a predetermined site in the lumen based on the endoscope image IMG. The predetermined site is a portion beyond the mid-trans in the sigmoid colon or transverse colon.

There are less painful insertion methods for the free colon, such as the shaft retention and shortening method. In such an insertion method, the insertion proceeds while maintaining an appropriate distance between the distal end section of the endoscope 10 and the intestinal wall by the suction. According to the present embodiment, the distance between the distal end section of the endoscope 10 and the intestinal wall is appropriately maintained by automatic suction control; therefore, the suction technique of an expert can be reproduced, or it allows the user to concentrate on the manipulation in the insertion.

If the determination section 160 detects, based on the endoscope image IMG, a red ball state in which the distal end section of the endoscope 10 is in contact with the mucosa of the subject, the determination section 160 determines not to perform the suction.

If the suction is performed in the red ball state, the mucosa or the intestinal wall may be damaged. According to the present embodiment, suction is turned off when a red ball state is detected, thus preventing the mucosa or the intestinal wall from being damaged by the suction.

If the determination section 160 determines, based on the endoscope image IMG, that the lumen is in an excessive air supply state in which the lumen has swelled beyond a predetermined level as a result of air supply, the determination section 160 determines to perform the suction.

The excessive air supply state may cause pain or discomfort in the patient. According to the present embodiment, suction is turned off in the excessive air supply state, thus preventing the patient from experiencing pain or discomfort due to excessive air supply.

The determination section 160 may acquire inspection status information, which is information regarding the status of the endoscopy using the endoscope 10, based on the endoscope image IMG, and determine whether or not the suction is needed based on the inspection status information. The inspection status information is information indicating the current inspection status, time-series inspection status including the current inspection status, or past inspection status. The inspection status information includes, for example, information indicating an insertion status of the endoscope, information indicating an operation status, information indicating a state of the intestine, and the like. As described later in the first detailed configuration example, the inspection status information is obtained, for example, as a combination pattern of a plurality of parameters indicating the inspection status, and the determination section 160 performs determination as to whether or not the suction is needed by referring to a control table in which the pattern is associated with suction control determination.

According to the present embodiment, the inspection status, including an insertion status of the endoscope, an operation status, and a state of the intestine, is recognized from the endoscope image. This enables appropriate automatic suction control according to the inspection status.

Although the case where the determination as to whether or not the suction is needed is performed as automatic suction control is described above, it is also possible to further perform suction strength determination. That is, the determination section 160 performs suction strength determination in which the suction strength is determined based on the endoscope image IMG. The control section 170 implements control to carry out the suction with the strength determined by the determination section 160. Specifically, the determination section 160 performs an image recognition process with respect to the endoscope image IMG to determine whether the suction should be strengthened or weakened in this situation, and based on the recognition result, outputs a determination signal DTR indicating a suction strength. For example, the determination section 160 may determine an insertion status by an image recognition process, and then perform the suction strength determination based on the insertion status. However, the determination section 160 may obtain the determination result regarding suction strength directly from the endoscope image IMG by an image recognition process, without determining the insertion status.

According to the present embodiment, the suction strength determination is performed based on the endoscope image, thus enabling appropriate automatic suction strength control according to the situation recognized from the endoscope image. This enables suction control, which has been difficult to achieve only by the control of making the internal pressure constant. The present embodiment is applicable to a variety of situations; however, as an example, the suction can be strengthened if a situation in which the distal end of the endoscope and the luminal inner wall are far from each other upon the insertion is recognized from the endoscope image. Further, if a situation where the distal end of the endoscope comes closer to the luminal inner wall by the suction is recognized from the endoscope image, the suction can be weakened.

As an example, the determination section 160 performs suction strength determination according to the distance between the mucosa of the subject and the distal end section of the endoscope 10 based on the endoscope image IMG. Specifically, the determination section 160 weakens the suction as the distance between the mucosa of the subject and the distal end section of the endoscope 10 becomes closer by the suction.

According to the present embodiment, such a control of suction strength allows for more appropriate control of the intestine, compared to the control of intestine by merely turning on or off the suction. This enables automatic suction control that more closely resembles the suction technique of an expert.

As another example, the determination section 160 recognizes at least one of a residue, water, a resected tissue, or a sprayed chemical liquid based on the endoscope image IMG, and performs determination as to whether or not the suction is needed and suction strength determination, according to the recognized result.

According to the present embodiment, if a residue, water, a resected tissue, or a sprayed chemical liquid is present, they can be suctioned out by turning on the suction. Further, the suction strength can be automatically controlled according to the amount of a residue, water, a resected tissue, or a sprayed chemical liquid.

Although the automatic suction control based on the endoscope image IMG is described above, the automatic suction control may be performed also using other items of information. As an example, the information acquisition section 140 may acquire insertion shape information, which is information representing the shape of the insertion section of the endoscope 10. The determination section 160 may determine whether or not the suction is needed based on the endoscope image IMG and the insertion shape information.

According to the present embodiment, the position and the shape of the insertion section or their changes can be directly determined by the determination of inspection status. For example, the site where the endoscope 10 is located, the operation being performed by the doctor, the amount of the operation, the procedures during the operation, the shape of the insertion section during the operation, and the like may be determined. This can improve the accuracy in the determination of inspection status.

Although the automatic suction control has been described above, automatic air supply control may also be performed as described later in the third detailed configuration example. That is, the determination section 160 may determine whether or not air supply is necessary based on the endoscope image IMG. The control section 170 may implement control to carry out air supply if the determination section 160 determines that air supply is necessary.

In order to minimize the burden on the patient during the inspection, control of the intestine or the like is performed by suction; however, under some circumstances, excessive suction may cause the intestine to collapse, thus making the operation difficult. According to the present embodiment, by performing automatic air supply control, it is possible to create the most suitable situation for the operation.

The process performed by the control device 100 described above may be performed as a control method in the following manner. The entity implementing the control method is not limited to the control device 100, but may be various systems or devices, such as the endoscope system described later. The control method includes: acquiring an endoscope image IMG, which is an image captured by the endoscope 10; performing determination as to whether the suction is needed so as to determine whether or not the suction is necessary based on the endoscope image IMG; and, when the suction is determined to be needed by the determination as to whether the suction is needed, implementing control to carry out the suction.

In addition, a part or all of the control device 100 described above may be implemented by a processor. In this case, the control device 100 may be configured as follows.

The control device 100 includes a memory for storing information and a processor that operates based on the information stored in the memory. The information includes, for example, a program and various types of data. The program describes a part or all of the functions of the information acquisition section 140, the determination section 160, and the control section 170. The processor implements a part or all of the functions of the information acquisition section 140, the determination section 160, and the control section 170 by executing the program.

The processor includes hardware, and the hardware may include at least one of a circuit that processes a digital signal and a circuit that processes an analog signal. For example, the processor may include one or a plurality of circuit devices or one or a plurality of circuit elements mounted on a circuit board. The one or a plurality of circuit devices is, for example, an integrated circuit (IC). The one or a plurality of circuit elements is, for example, a resistor or a capacitor. For example, the processor may be a central processing unit (CPU). However, the processor is not limited to the CPU, and various processors such as a Graphics Processing Unit (GPU), a Digital Signal Processor (DSP), and the like, may be used. The processor may also be an integrated circuit device, such as an Application Specific Integrated Circuit (ASIC), a Field Programmable Gate Array (FPGA), or the like. The processor may include an amplifier circuit, a filter circuit, or the like that processes an analog signal. The memory may be a semiconductor memory such as an SRAM, a DRAM or the like, or may be a register. The memory may also be a magnetic storage device, such as a hard disk device or an optical storage device such as an optical disc device. For example, the memory stores therein a computer-readable commands, and the processes (functions) of the sections of the control device 100 are implemented with the processor executing the commands. These commands may be a command set included in a program, or may be commands to give operating instructions to the hardware circuit of the processor.

The program described above may be stored in a non-transitory information storage medium, which is a computer-readable medium. The information storage medium can be implemented by, for example, an optical disc, a memory card, an HDD, a semiconductor memory, or the like. The semiconductor memory is, for example, a ROM or a nonvolatile memory.

### 2. First Detailed Configuration Example

In the following, an example of applying the control device 100 to endoscopy of the large intestine is described. However, the application of the control device 100 is not limited to endoscopy of the large intestine.

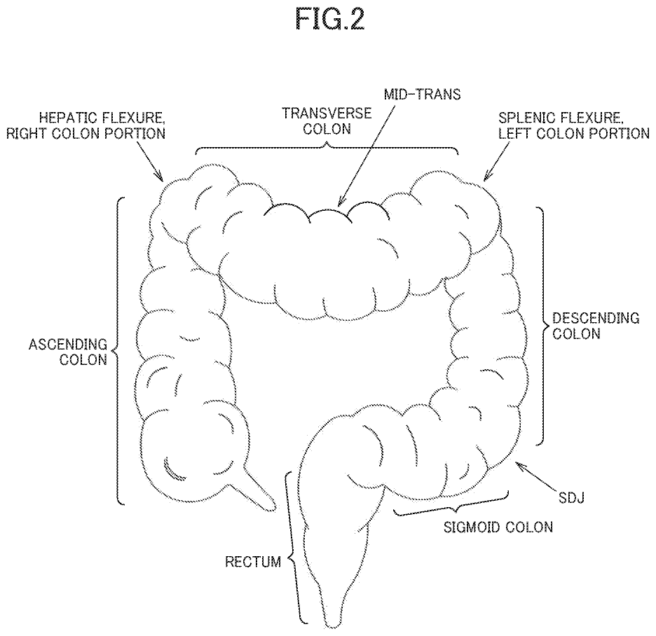

FIG. 2 is an explanatory view of parts of the large intestine. The parts of the large intestine are, from the cecum to the anus, the ascending colon, hepatic flexure, transverse colon, splenic flexure, descending colon, SDJ, sigmoid colon, and rectum. The area near the hepatic flexure is also called a right colon portion, and the area near the splenic flexure is also called a left colon portion. SDJ stands for Sigmoid Descending colon Junction, which is the boundary between the sigmoid colon and the descending colon. The ascending colon, the descending colon, and the rectum are fixed colons, while the transverse colon and the sigmoid colon are free colons.

In endoscopy, very high techniques are required to perform high-quality endoscopy with minimal burden on the patient. One of the important endoscope operations to perform such an endoscopy is suction operation. The suction operation serves not only to suck air and water from the intestinal tract, but also to (1) recognize the state of the intestine and (2) control the intestine.

As an example of (1), if the lumen appears to have an earthen tubular shape in the sigmoid colon, suction is first performed to recognize whether or not the intestine comes closer by the suction. If the intestine comes closer, the shaft retention and shortening method in which insertion is performed while shortening the intestine is selected; if the intestine does not come closer, the loop method in which the insertion is performed with a pushing operation of the scope is selected.

As an example of (2), when the endoscope is inserted across the folds while shortening the sigmoid colon, the endoscope moves forward while performing the suction, thereby bringing the distal end of the endoscope and the intestinal wall relatively close. This prevents the intestinal wall from moving away or being stretched by a pushing operation, a torque operation, or an angle operation. Furthermore, in controlling the intestine, experienced doctors minutely adjust the suction amount by pressing the suction button all the way down, as well as pressing it half or a quarter of the way down.

The suction operations for these purposes are very difficult for young doctors. The control device 100 of the present embodiment assists the suction function so that even younger doctors can achieve the same operation as those of the experienced doctors, thus providing a high-quality inspection with less burden on the patient. In endoscopy of the large intestine, the endoscope is first inserted from the rectum to the cecum, and then the intestinal tract is inflated by supplying air, and observation is performed while removing the endoscope. The control device 100 of the present embodiment may perform suction control during both insertion and observation, or only during insertion.

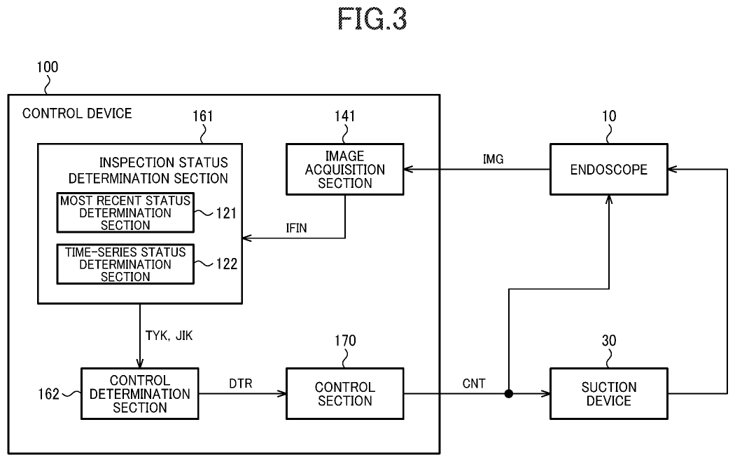

A detailed configuration example of the control device 100 of the present embodiment is detailed below. FIG. 3 illustrates a first configuration example of the control device 100. The control device 100 includes an image acquisition section 141, an inspection status determination section 161, a control determination section 162, and the control section 170. In this configuration example, the image acquisition section 141 corresponds to the information acquisition section 140 in FIG. 1, and the inspection status determination section 161 and the control determination section 162 correspond to the determination section 160 in FIG. 1. The same components as those already described will be omitted from the description as appropriate.

The image acquisition section 141 receives an endoscope image IMG transmitted by the endoscope 10, thereby acquiring the endoscope image IMG. The endoscope image IMG is input to the inspection status determination section 161 as information IFIN. The information of suction status acquired from the suction device may be further input to the inspection status determination section 161 as information IFIN.

The inspection status determination section 161 determines a most recent status and a time-series status from the information IFIN and outputs most recent status information TYK and time-series status information JIK. The inspection status determination section 161 includes a most recent status determination section 121 and a time-series status determination section 122.

The most recent status determination section 121 recognizes the most recent status of endoscope insertion and intestine status from the information IFIN, and outputs the result as the most recent status information TYK. The most recent status is the status recognized from a single endoscope image IMG of a moving image captured by the endoscope 10.

The time-series status determination section 122 recognizes the time-series status of endoscope insertion and intestine status from the information IFIN, and outputs the result as the time-series status information J1K. The time-series status is the status recognized from a plurality of time-series endoscope images IMG of a moving image captured by the endoscope 10.

The control determination section 162 determines the control of suction operation based on either or both of the most recent status information TYK and the time-series status information JIK. The control of suction operation is control of at least one of turning on/off of the suction, the suction strength, or switching of the suction path. The switching of the suction path means switching between an automatic suction mode in which suction is automatically controlled based on the information from the time-series status determination section 122, and a manual suction mode in which the doctor manually performs the suction operation. A suction button is provided on the endoscope 10, and the control determination section 162 switches from the automatic suction mode to the manual suction mode when intentional pressing of the suction button by the doctor is detected. In the manual suction mode, automatic suction control by the control device 100 is disabled and the suction device 30 is operated by manual control by the suction button.

As shown above, the control device 100 recognizes the current inspection status and intestine status from the endoscope image IMG, and assists the insertion by adjusting whether or not to perform suction, or adjusting whether or not to perform suction together with the suction amount, according to the status. In endoscopy, the suction is involved in the control of intestine, determination of the operation plan, removal of residues, or prevention of excessive air supply during the insertion, and is also involved in the removal of water, removal of chemical liquids, or removal of tumors during the observation or treatment. The control of intestine is such as prevention of extension of the free colon, prevention of pain, or the like. By assisting these suction operations, the control device 100 can provide a more painless and appropriate inspection for the patients, as well as a less burdensome inspection environment for the doctors.

As an example, the control device 100 determines the distance between the distal end of the endoscope and the intestinal wall based on the endoscope image during the shaft retention and shortening method, and performs automatic suction in a manner in which the distance is optimized. This enables the optimum distance between the distal end of the endoscope and the intestinal wall to be maintained. Further, the control device 100 not only performs suction at an appropriate timing, but also adjusts the suction amount automatically. This enables automatic suction control that more closely resembles the suction operation of an expert, compared to the control by merely turning on or off the suction. The control device 100 also performs adjustment as to whether or not to perform suction, or adjustment as to whether or not to perform suction together with the suction amount, not only during the shaft retention and shortening method, but also in various other situations where suction is needed. The various situations where suction is needed are, for example, a situation in which intestinal fluid is accumulated, a situation with excessive air supply, and the like. This not only maintains the optimum distance between the distal end of the endoscope and the intestinal wall during the shaft retention and shortening method, but also enables automatic suction control in various situations where suction is needed.

In the above-mentioned Japanese Unexamined Patent Application Publication No. H05-245100, a pressure sensor is provided in the forceps channel, and the internal celomic pressure adjusting device of the endoscope automatically performs suction according to the output value of the pressure sensor, thereby maintaining the internal celomic pressure at a constant level. The present embodiment performs determination as to whether or not the suction is needed, or determination as to whether or not the suction is needed together with the suction strength, based on the endoscope image, instead of using the output value of the pressure sensor. This enables automatic suction control even in situations where suction is needed but the need of suction cannot be detected from the output value of the pressure sensor, for example, a situation related to the optimum distance between the distal end of the endoscope and the intestinal wall during the shaft retention and shortening method, or a situation where intestinal fluid has accumulated, and the like.

The suction operation is an essential part of endoscopy of the large intestine, and automatic adjustment of the suction timing and suction amount is expected to maintain an appropriate state of the intestine, thus reducing the pain in the patients and improving the quality of the inspection. For example, during the insertion into the free colon, the intestine is drawn by the suction and an appropriate distance between the distal end of the endoscope and the intestinal wall is maintained, thereby reducing the operation that induces pains in the patient. In addition, the automatic suction allows the doctor to concentrate on other operations and also prevents pain in the patients caused by air supply. Also, when the intestinal tract is inflated by air supply upon the removal, the automatic suction control allows the intestinal tract to be maintained in a condition suitable for observation and painless for the patient, thus enabling high-quality observation. Further, when the observation is completed, it is possible to assist the observation and reduce discomfort of the patient by sucking the air in the intestine.

As an example, the inspection status determination section 161 is implemented by machine learning using a neural network or the like. More specifically, the memory (not shown) stores a program that describes an inference algorithm and parameters used for the inference algorithm, as the information of the trained model. Then, the processor performs the processing based on the information of the trained model. That is, the processor executes the program using the parameters stored in the memory, thereby executing the processing of the inspection status determination section 161. The entire inspection status determination section 161 may be implemented by a single trained model, or each of the most recent status determination section 121 and the time-series status determination section 122 may be implemented by individual trained models.

For example, a neural network may be used as the inference algorithm. The weight coefficients of the inter-node connections in the neural network correspond to the parameters. The neural network includes an input layer to which input data is entered, an intermediate layer for performing a calculation process with respect to the data entered via the input layer, and an output layer for outputting a recognition result based on the calculation result output from the intermediate layer. The inference algorithm is not limited to a neural network, and various types of machine learning techniques for use in recognition process may be used. The input data is the information IFIN, and the recognition results are the most recent status information TYK and the time-series status information JIK. The training device that executes the learning process is, for example, an information processing device such as a PC. The training device generates a trained model by inputting training data into a training model, and providing feedback to the training model based on the recognition result. The training data contains multiple sets of data, each set containing input data and correct answer data. The correct answer data is the recognition result that is supposed to be obtained in response to the input data. The correct answer data is prepared in advance, for example, by a medical worker.

Not only the inspection status determination section 161 but also the control determination section 162 may be implemented by machine learning. In this case, the recognition result output from the output layer of the neural network is the determination signal DTR. Alternatively, the recognition result may be the determination signal DTR, the most recent status information TYK, and the time-series status information JIK.

An example of a case where the control determination section 162 performs control determination by an algorithm other than machine learning is a method using a control table. Specifically, the control determination section 162 stores a control table in which combination patterns of a plurality of parameters indicating the inspection status are associated with suction control determination. The inspection status determination section 161 outputs a determination result for each parameter from the information IFIN, and the control determination section 162 performs suction control determination by referring to the control determination results corresponding to the determination results for the plurality of parameters output by the inspection status determination section 161 from the table.

The plurality of parameters indicating the inspection status correspond to the most recent status information TYK and the time-series status information JIK, and indicate, for example, the site where the distal end of the endoscope is located, the distance between the distal end of the endoscope and the intestinal wall, changes in the distance, the endoscope insertion method currently performed, the endoscope operation currently performed, presence or absence of adhesion, presence or absence of water, presence or absence of residues, or whether or not a red ball state is present, and the like. For example, in the control table, control determination to turn on the suction is associated with a parameter pattern in which the site is the sigmoid colon, the insertion method is the shaft retention and shortening method, and the distance is greater than the predetermined distance. However, the association is not limited to this, and the control table is configured so that the desired suction control is achieved.

### 3. Second Detailed Configuration Example

A specific example of automatic suction control is described below as a second detailed configuration example. The configuration of the control device 100 is the same as that in FIG. 3.

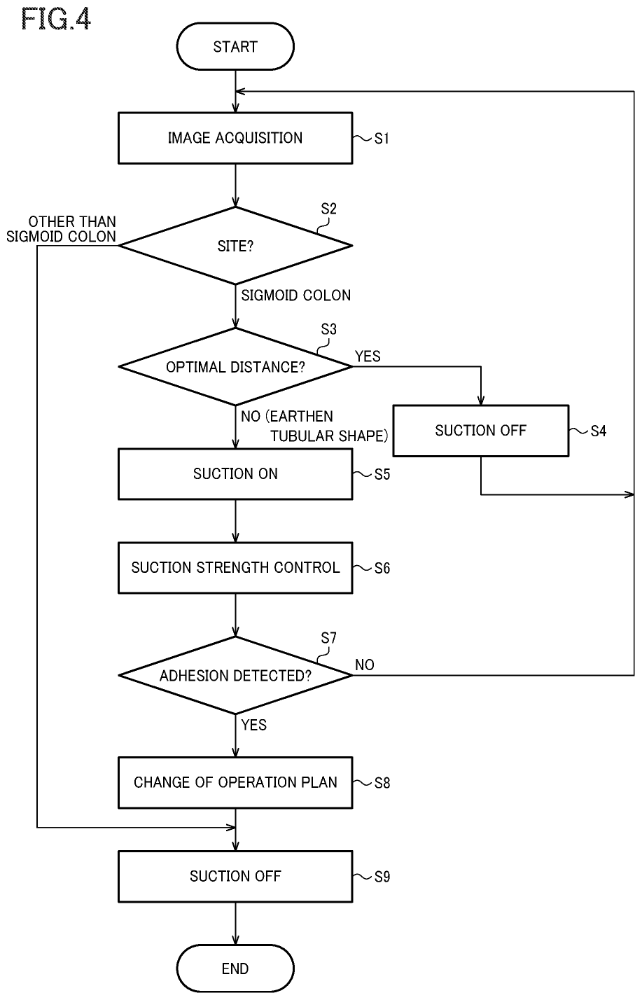

FIG. 4 is a flowchart showing an example of automatic suction control when performing the shaft retention and shortening method in the sigmoid colon.

In the step S1, the image acquisition section 141 acquires an endoscope image IMG from the endoscope 10. In the step S2, the inspection status determination section 161 determines the site from the endoscope image IMG. The inspection status determination section 161 recognizes the site from the endoscope image IMG by using the fact that the characteristics of the image differ depending on the site. When the insertion shape information, which is described later, is used, the inspection status determination section 161 may acquire the length of a portion of the endoscope insertion section being inserted into the patient from the insertion shape information and may presume the site from that insertion length.

If the inspection status determination section 161 determines in the step S2 that the endoscope 10 is located in a region other than the sigmoid colon, in the step S9, the control determination section 162 determines to turn off the suction, and the control section 170 outputs the control signal CNT to turn the suction off.

If the inspection status determination section 161 determines in the step S2 that the endoscope 10 is located in a region other than the sigmoid colon, in the step S3, the inspection status determination section 161 determines from the endoscope image IMG whether or not the distance between the distal end section of the endoscope 10 and the intestinal wall is the optimal distance. The optimal distance refers to the optimal distance between the insertion section and the intestinal wall with which the folds can be flipped by either or both of the angle operation and the torque operation.

If the inspection status determination section 161 determines in the step S3 that the distance between the distal end section of the endoscope 10 and the intestinal wall is the optimal distance, in the step S4, the control determination section 162 determines to turn off the suction, and the control section 170 outputs the control signal CNT to turn the suction off. Then, the process returns to the step S1.

When the inspection status determination section 161 determines in the step S3 that the distance between the distal end section of the endoscope 10 and the intestinal wall is not the optimal distance, in the step S5, the control determination section 162 determines to turn on the suction, and the control section 170 outputs the control signal CNT to turn the suction on.

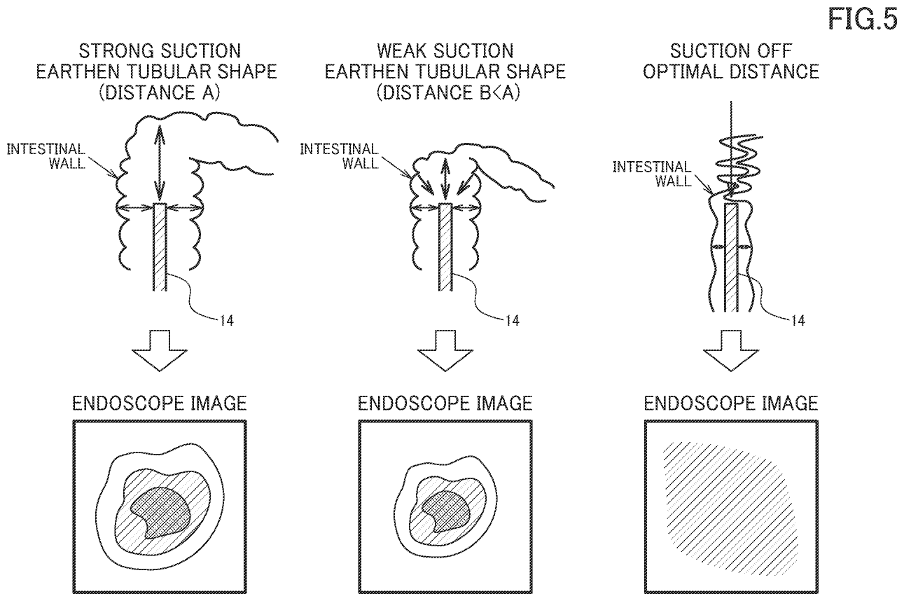

In the step S6, the control determination section 162 performs suction strength determination. FIG. 5 shows a first example of suction strength determination. The control determination section 162 determines that the suction is gradually weakened as the distance between the distal end section of the insertion section 14 and the intestinal wall comes closer to the optimal distance, and the control section 170 outputs the control signal CNT to specify the suction amount according to the determination result. FIG. 5 shows an example of suction strength control in two stages, in which the suction is set high in the case of the distance A with the earthen tubular shape, and the suction is set low in the case of the distance B, which is smaller than the distance A, with the earthen tubular shape. As shown in FIG. 5, the suction causes the intestinal tract to fold into a deflated shape, and the appearance of the intestinal tract in the image changes depending on the degree of deflation. In the case of the earthen tubular shape, a luminal-shaped intestinal tract appears. With the optimal distance, a folded intestinal wall appears. The distance between the distal end section of the insertion section 14 and the intestinal wall can be presumed from these appearances of the intestinal tract. For example, the distance is the distance between the distal end of the insertion section and the intestinal wall in the optical axis direction of the imaging section. Alternatively, the distance may be comprehensively determined from the distance between the distal end of the insertion section and the intestinal wall in various directions. If the optimal distance is achieved by the suction, the suction is turned off in the step S4.

In the step S7, the inspection status determination section 161 determines the presence or absence of adhesions from the changes in the endoscope image IMG during the suction. The presence or absence of adhesions can be recognized from the image because the adhesion portion of the intestinal tract is unlikely to change by the suction.

If the inspection status determination section 161 determines that there is no adhesion in the step S7, the process returns to the step S1.

If the inspection status determination section 161 determines that there is adhesion in the step S7, the inspection status determination section 161 changes the operation plan from the shaft retention and shortening method to the loop method in the step S8. In the step S9, in response to the change of the operation plan to the loop method, the control determination section 162 determines to turn off the suction.

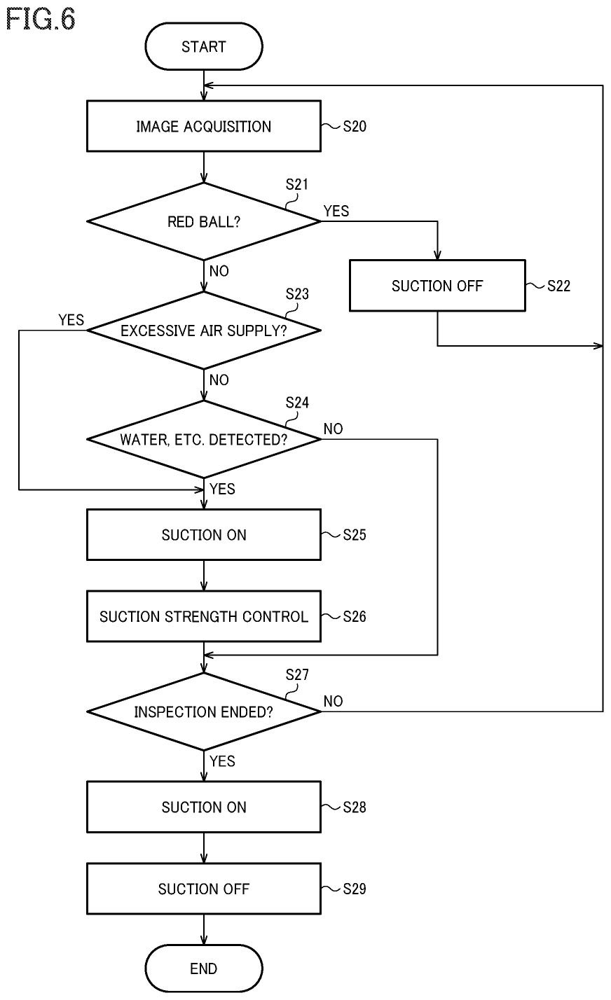

FIG. 6 is a flowchart showing an example of automatic suction control based on inspection status other than the distance.

In the step S20, the image acquisition section 141 acquires the endoscope image IMG from the endoscope 10. In the step S21, the inspection status determination section 161 determines from the endoscope image IMG whether or not a red ball state is present. The red ball state is a state in which a part or all of the image appears reddish due to contact of the objective lens on the distal end section of the endoscope 10 with the mucosa, and the inspection status determination section 161 can recognize the red ball state from that image.

If the inspection status determination section 161 determines in the step S20 the presence of a red ball state, in the step S22, the control determination section 162 determines to turn off the suction, and the control section 170 outputs the control signal CNT to turn the suction off. Then, the process returns to the step S20.

If the inspection status determination section 161 determines in the step S20 the absence of the red ball state, in the step S23, the inspection status determination section 161 determines whether or not the air supply is excessive. Excessive air supply can be recognized by the way the image appears depending on the thickness of the lumen or by extended folds shown in the image.

If the inspection status determination section 161 determines in the step S23 that the air supply is excessive, in the step S25, the control determination section 162 determines to turn on the suction, and the control section 170 outputs the control signal CNT to turn the suction on.

If the inspection status determination section 161 determines in the step S23 that the air supply is not excessive, in the step S24, the inspection status determination section 161 determines the presence or absence of water or other substances. The water and other substances refer to water, residues, chemical liquids, or tissue fragments. The tissue fragments are polyps, tumors, and the like, that have been resected during the observation or treatment. The water or chemical liquid is injected into the lumen, for example, by a syringe through a port provided in the endoscope. Alternatively, water is injected into the lumen by water jet controlled by a footswitch.

If the inspection status determination section 161 determines in the step S24 the presence of water, residues, or tissue fragments, in the step S25, the control determination section 162 determines to turn on the suction, and the control section 170 outputs the control signal CNT to turn the suction on.

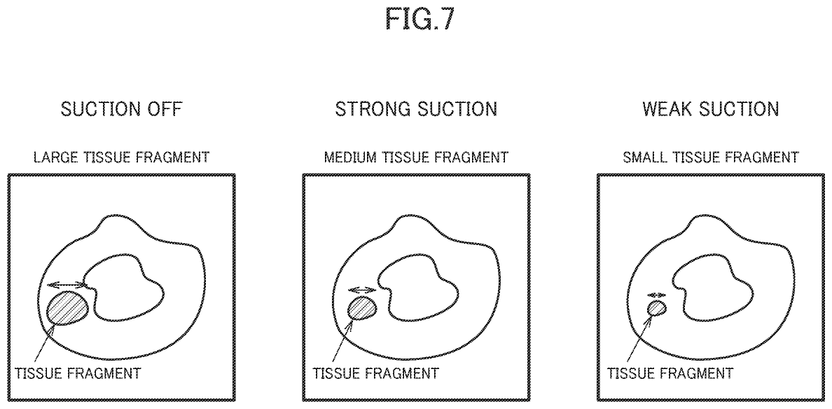

In the step S26, the control determination section 162 performs suction strength determination. FIG. 7 shows a second example of suction strength determination. The inspection status determination section 161 recognizes the presence of a tissue fragment and the size of the tissue fragment from the endoscope image. The control determination section 162 determines to strengthen the suction as the size of the tissue fragment recognized by the inspection status determination section 161 increases. The control section 170 outputs the control signal CNT to specify the suction amount according to the determination result. If the size of the tissue fragment is larger than a predetermined size, the control determination section 162 determines to turn off the suction because the tissue fragment cannot pass through the suction tube. As another example of suction strength determination, the control determination section 162 determines to strengthen the suction as the amount of water or residue shown in the endoscope image increases.

After the step S26 or in the step S24, if the control determination section 162 determines the absence of water, residues, or tissue fragments, in the step S27, the inspection status determination section 161 determines whether or not the inspection has been completed. The inspection status determination section 161 determines that the inspection has been completed based on input information from the doctor or when a predetermined state is recognized from the endoscope image. The predetermined state is a state in which the endoscope 10 has been removed to the rectum or a state in which the endoscope 10 has been taken out from the body.

If the inspection status determination section 161 determines in the step S27 that the inspection has not been completed, the process returns to the step S20.

If the inspection status determination section 161 determines in the step S27 that the inspection has been completed, in the step S28, the control determination section 162 determines to turn on the suction, and the control section 170 outputs the control signal CNT to turn the suction on. In the step S29, after the inflation of the intestinal tract caused by the air supply is released by the suction, the control determination section 162 determines to turn off the suction, and the control section 170 outputs the control signal CNT to turn the suction off.

As described above, the control device 100 of the present embodiment is capable of preventing extension of the intestine or pain by attracting the intestinal wall by suction when the endoscope 10 is inserted into the intestine in a non-fixed portion. Further, the control device 100 improves the insertion property by preventing red balls by the suction control or by adjusting the amount of air. The control device 100 can also improve the insertion property by performing automatic suction control when it is determined that the presence of water, residues, or the like is blocking a clear view, thus causing problems with the insertion, when the endoscope is inserted into the intestine of a non-fixed portion or fixed portion.

The suction control described above is an example. As another example, when the inspection status determination section 161 determines that the distal end section of the endoscope 10 has crossed the mid-trans during the transverse colon mid-trans operation, the control determination section 162 determines to turn on the suction. The suction brings the intestinal wall closer to the distal end section of the endoscope 10, ensuring a suitable distance for mid-trans operation.

### 4. Third Detailed Configuration Example

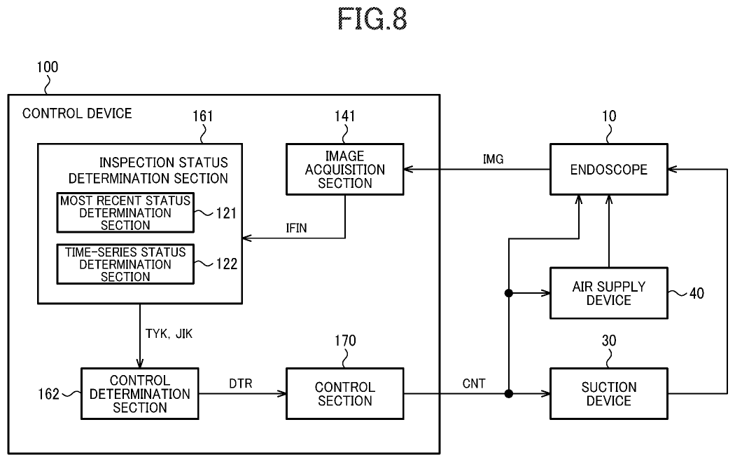

FIG. 8 illustrates a third detailed configuration example of the control device 100. In this configuration example, the control device 100 performs not only the automatic suction control but also automatic air supply control. As the automatic suction control was described above, automatic air supply control is mainly described below.

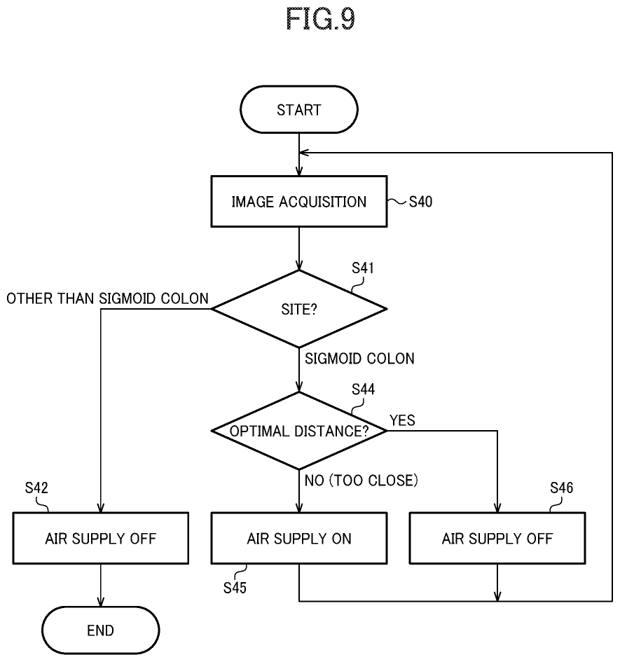

FIG. 9 is a flowchart showing an example of automatic air supply control.

In the step S40, the image acquisition section 141 acquires an endoscope image IMG from the endoscope 10. In the step S41, the inspection status determination section 161 determines the site from the endoscope image IMG.

If the inspection status determination section 161 determines in the step S41 that the endoscope 10 is located in a region other than the sigmoid colon, in the step S42, the control determination section 162 determines to turn off the air supply, and the control section 170 outputs the control signal CNT to turn the air supply off. An air supply device 40 turns off the air supply in response to the control signal CNT. Although the control signal for air suction and the control signal for air supply are collectively referred to as CNT, air suction and air supply are independently controlled.

If the inspection status determination section 161 determines in the step S41 that the endoscope 10 is located in a region other than the sigmoid colon, in the step S44, the inspection status determination section 161 determines from the endoscope image IMG whether or not the distance between the distal end section of the endoscope 10 and the intestinal wall is the optimal distance.

If the inspection status determination section 161 determines in the step S44 that the distance between the distal end section of the endoscope 10 and the intestinal wall is the optimal distance, in the step S46, the control determination section 162 determines to turn off the air supply, and the control section 170 outputs the control signal CNT to turn the air supply off. An air supply device 40 turns off the air supply in response to the control signal CNT. Then, the process returns to the step S1.

If the inspection status determination section 161 determines in the step S44 that the distance between the distal end section of the endoscope 10 and the intestinal wall is not the optimal distance, in the step S45, the control determination section 162 determines to turn on the air supply, and the control section 170 outputs the control signal CNT to turn the air supply on. The air supply device 40 turns on the air supply in response to the control signal CNT. Then, the process returns to the step S1.

In order to minimize the burden on the patient during the inspection, control of the intestine is performed by suction; however, under some circumstances, excessive suction may cause the intestine to collapse, thus making the operation difficult. In the present embodiment, by performing automatic air supply control, it is possible to create the most suitable situation for the operation.

### 5. Endoscope, Suction Device, and Air Supply Device

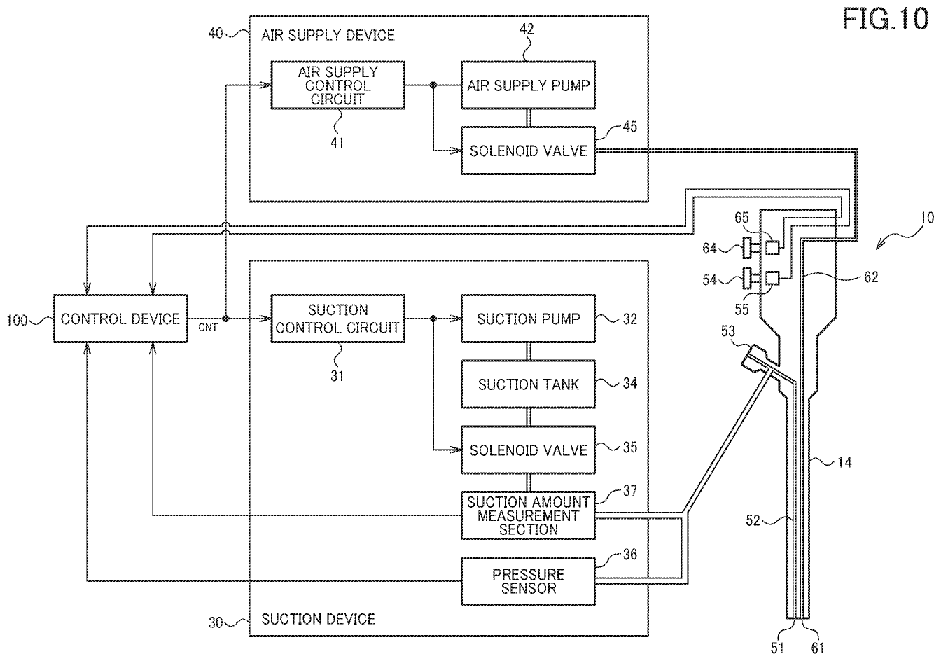

FIG. 10 illustrates detailed configuration examples of the endoscope 10, the suction device 30, and the air supply device 40. FIG. 10 omits, where appropriate, illustration of configurations not involved in suction and air supply. For example, the illumination device, the imaging device, the angle operating dial, etc. of the endoscope 10 are not shown in FIG. 10. The configuration shown in FIG. 10 is an example, and the configurations of the endoscope 10, the suction device 30, and the air supply device 40, and the connection relationship among the components are not limited to those shown in FIG. 10.

The endoscope 10 includes a suction operation button 54, a suction operation detection sensor 55, an air supply operation button 64, an air supply operation detection sensor 65, a port 53, an insertion section 14, a suction port 51, a suction tube 52, an air supply port 61, and an air supply tube 62.

The suction tube 52 is provided in the insertion section 14, one end of which is connected to the suction port 51 provided at the distal end of the insertion section 14, while the other end is connected to the port 53. The port 53 is provided for allowing treatment tools such as forceps to be inserted into the body cavity from the suction port 51 via the suction tube 52, or for dispersing chemical liquids and the like into the body cavity from the suction port 51 via the suction tube 52 using a syringe. The suction tube 52 branches near the port 53, and the branched suction tube 52 is connected to the suction device 30.

The air supply tube 62 is provided in the insertion section 14, the endoscope grip section, and the universal cord, one end of which is connected to the air supply port 61 provided at the distal end of the insertion section 14, while the other end is connected to the air supply device 40.

The suction operation button 54, the air supply operation button 64, the suction operation detection sensor 55, and the air supply operation detection sensor 65 are provided in the endoscope grip section. The suction operation detection sensor 55 detects the pressing of the suction operation button 54, and outputs a detection signal to the control determination section 162. The air supply operation detection sensor 65 detects the pressing of the air supply operation button 64, and outputs a detection signal to the control determination section 162.

The suction device 30 includes a suction control circuit 31, a suction pump 32, a suction tank 34, a solenoid valve 35, a suction amount measurement section 37, and a pressure sensor 36.

The suction tube 52, which branches near the port 53, is connected to the suction pump 32 via the suction amount measurement section 37, the solenoid valve 35, and the suction tank 34. The suction control circuit 31 includes a drive circuit for the suction pump 32 and a drive circuit for the solenoid valve 35, and controls ON/OFF of the suction as well as the suction strength by driving the suction pump 32 and the solenoid valve 35 according to the control signal CNT output from the control section 170. The suction amount measurement section 37 measures the amount of gas or liquid sucked by the suction pump 32, and outputs the measurement value to the inspection status determination section 161. The suction amount measurement section 37 is, for example, a flow rate sensor.

The pressure sensor 36 is connected to the suction tube 52, which branches near the port 53. The pressure sensor 36 measures the pressure in the suction tube 52, and outputs the measurement value to the inspection status determination section 161.

The air supply device 40 includes an air supply control circuit 41, an air supply pump 42, and a solenoid valve 45.

The air supply tube 62 is connected to the air supply pump 42 via the solenoid valve 45. The air supply control circuit 41 includes a drive circuit for the air supply pump 42 and a drive circuit for the solenoid valve 45, and controls ON/OFF of the air supply by driving the air supply pump 42 and the solenoid valve 45 according to the control signal CNT output from the control section 170.

In switching the path, when the control determination section 162 recognizes that the suction operation button 54 has been operated based on the detection signal from the suction operation detection sensor 55, the control determination section 162 switches the automatic suction mode to the manual suction mode. In the manual suction mode, the control section 170 outputs the detection signal from the suction operation detection sensor 55 as the control signal CNT to the suction control circuit 31.

Furthermore, when the control determination section 162 recognizes that the air supply operation button 64 has been operated based on the detection signal from the air supply operation detection sensor 65, the control determination section 162 may switch the automatic air supply mode to the manual air supply mode. In the manual air supply mode, the control section 170 outputs the detection signal from the air supply operation detection sensor 65 as the control signal CNT to the air supply control circuit 41. In the first and second detailed configuration examples, switching of air supply path may be omitted, and the configurations may have only the manual air supply mode. That is, the detection signal from the air supply operation detection sensor 65 may be input directly to the air supply control circuit 41.

### 6. Fourth Detailed Configuration Example

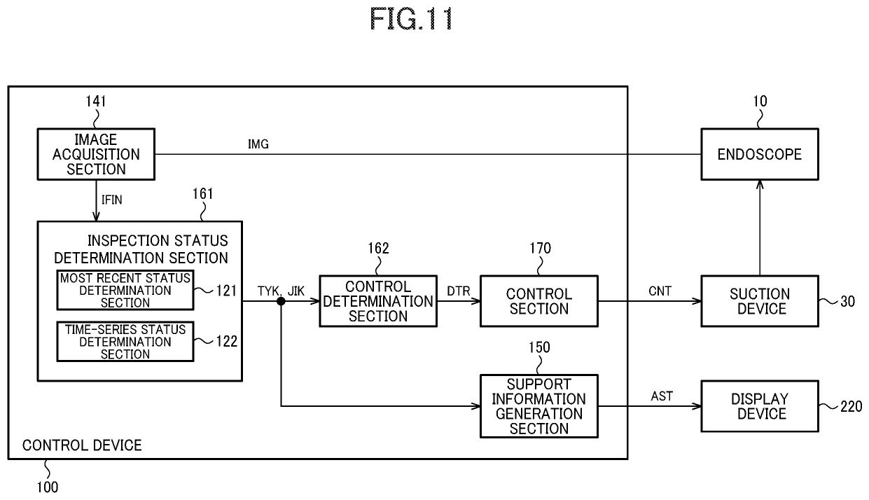

FIG. 11 illustrates a fourth detailed configuration example of the control device 100. In this configuration example, the control device 100 further includes a support information generation section 150. As the automatic suction control was described above, support information generation is mainly described below. As in the third detailed configuration example, automatic air supply control may further be used in combination.

The support information generation section 150 generates support information AST according to the information IFIN. The support information AST is information to support insertion of an endoscope into the patient. Specifically, the support information AST is information to be presented or notified to the medical worker or insertion/removal device that performs the operation of inserting the endoscope into the patient, and indicates what operation should be performed next for the current insertion status, or it is information to allow a doctor to grasp the current insertion status, or the like. The support information generation section 150 determines the endoscope insertion status from the most recent status information TYK and the time-series status information JIK, and generates the support information AST according to the insertion status. An insertion status is a predetermined insertion status that appears in the procedure to proceed with the endoscopy, and is identified, for example, by a predetermined position of the insertion section, a change in the predetermined position of the insertion section, a predetermined shape of the insertion section, a change in the predetermined shape of the insertion section, a predetermined operation of the endoscope, or any combination of two or more of them. The information regarding the shape of the insertion section, the change in the shape of the insertion section, and the operation of the endoscope may be acquired by insertion shape information, or the like, which is described later.

FIG. 11 illustrates an example in which the support information generation section 150 displays text, symbols, images, or the like, as the support information AST in the display device 220. However, the support information AST is not limited to images, and may also be presented in the form of sound, voice, light, vibration, or the like. Alternatively, if an automatic insertion/removal device is used, a control signal that causes the automatic insertion/removal device to perform operation or processes may be input to the automatic insertion/removal device as the support information AST. The presentation of the support information AST and the automatic suction control may be performed at the same time, or only one of the presentation of the support information AST and the automatic suction control may be performed depending on the situation.

A specific example of the support information presentation is described below. There are insertion methods for performing insertion into non-fixed portions of the intestine, such as the sigmoid colon or transverse colon, and, in each insertion method, there are insertion statuses, such as the shape of the insertion section or the operation, that appear when proceeding with the insertion procedure. In each of those insertion statuses, the next operation to be performed is fixed. The support information generation section 150 generates the support information AST according to the insertion procedure.

In the present embodiment, when the insertion is made to reach a non-fixed portion of the intestine, such as the sigmoid colon or transverse colon, not only the intestine control by automatic suction, but also the presentation of operation support information, can be performed. This will further improve the insertion property and enable insertion support. The support information may be generated and presented using information of the situation that is found out by the suction. The situation that is found out by the suction may be, for example, the presence or absence of adhesions or what insertion methods may be used, and the support information presentation may include an insertion plan or the like according to the situation. The insertion methods that can be used include, for example, the loop method, the shaft retention and shortening method, and the like.

As a first example of support information presentation, the control device 100 performs the following presentation of the support information AST or automatic suction control during an endoscope operation in the sigmoid colon. When the inspection status determination section 161 determines that a suction operation, such as the shaft retention and shortening method, is necessary, the control determination section 162 and the control section 170 perform automatic suction, and the support information generation section 150 presents operation support information. When the inspection status determination section 161 recognizes that the endoscope 10 has entered to the sigmoid colon, the support information generation section 150 presents a suction operation, or the control determination section 162 and the control section 170 perform suction automatically.

Further, the support information generation section 150 changes the content of the next support information AST to be presented based on the status of the suction. If the intestinal wall comes closer by the suction so that the optimal distance can be maintained, the support information generation section 150 presents a guide of the shaft retention and shortening method. Until the earthen tubular shape becomes the optimal distance by the suction, the support information generation section 150 presents a guide for the suction operation in addition to the shaft retention operation. At this time, automatic suction may be performed. If a red ball state is present, the support information generation section 150 presents the cessation of the suction, or the control determination section 162 and the control section 170 automatically stop the suction. If the intestinal wall does not come closer even by the suction, the support information generation section 150 presents a guide of the Push method, such as the N-loop method. The support information generation section 150 successively changes the content of the guide according to the situation at that time.

As a second example of the support information presentation, when the inspection status determination section 161 determines that the operation is an operation requiring control of intestine by the suction, such as a transverse colon shortening operation, the control determination section 162 and the control section 170 perform automatic suction, and the support information generation section 150 presents the operation support information. When the inspection status determination section 161 determines that the endoscope has passed the mid-trans, the support information generation section 150 presents the suction operation, or the control determination section 162 and the control section 170 performs the suction automatically.

Further, the support information generation section 150 changes the content of the next support information AST to be presented based on the status of the suction. If the intestinal wall comes closer by the suction, and the optimal distance can be maintained, the support information generation section 150 presents a shortening operation guide using suction. If the intestinal wall does not come closer even by the suction, the support information generation section 150 presents a shortening operation guide that does not use suction. The support information generation section 150 successively changes the content of the guide according to the situation at that time.

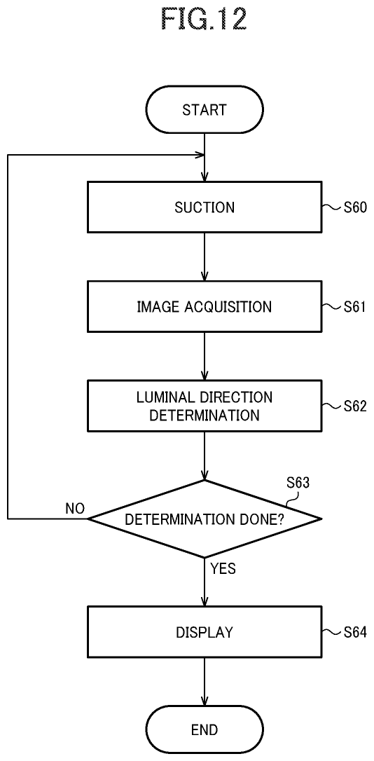

As a third example of support information presentation, presentation of luminal direction may be performed. The support information generation section 150 performs dedicated presentation of luminal direction depending on the presence or absence of the suction. FIG. 12 is a flowchart showing an example of luminal direction presentation.

In the step S60, suction is performed. This suction may be performed by automatic suction control or by manual operation. In the step S61, the image acquisition section 141 acquires an endoscope image IMG from the endoscope 10.

In the step S62, the inspection status determination section 161 determines the luminal direction from the endoscope image IMG. In the step S63, it is determined whether or not the luminal direction has been determined. If the luminal direction cannot be determined, the process returns to the step S60, and if the luminal direction can be determined, the support information generation section 150 displays the determined luminal direction on the display device 220 in the step S64.

During the operation accompanying suction, the endoscope image IMG tends to be a lumen image peculiar to that during the suction or a red ball state, thus making it difficult to find the direction of the lumen. Therefore, a trained model that has been trained with endoscope images of lumen at the time of suction is generated, and the inspection status determination section 161 performs lumen recognition exclusively for suction by an inference process using the trained model. The inspection status determination section 161 then presumes and tracks the luminal direction using the recognition result and optical flow technology or the like, and the support information generation section 150 successively presents the resulting luminal directions on the screen. In lumen tracking, the inspection status determination section 161 may presume the luminal direction using images such as optical flow, or may presume the luminal direction using endoscope movements obtained from insertion shape information such as UPD, or may presume the luminal direction based on information acquired from a sensor attached to the doctor. The UPD and the sensor attached to the doctor are described later. Upon the suction, moisture in the intestine is also sucked. Therefore, the inspection status determination section 161 may presume the flow of water due to suction from the endoscope image and predict the luminal direction from the presumption result, and the support information generation section 150 may present the luminal direction thus predicted.

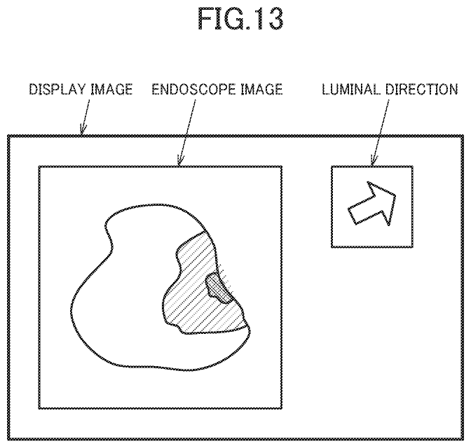

FIG. 13 shows an example of a luminal direction presentation screen. The support information generation section 150 causes the display device 220 to display an arrow indicating the luminal direction together with the endoscope image as a display image. The arrow indicating the luminal direction corresponds to the support information AST. Although FIG. 13 separately shows the endoscope image and the arrow, the arrow may be superimposed on the endoscope image.

As another example of the support information presentation, the inspection status determination section 161 determines that there is a possibility of excessive air supply if the lumen is open, and the support information generation section 150 presents suction, or the control determination section 162 and the control section 170 perform suction automatically.

Further, the inspection status determination section 161 determines the presence or absence of adhesion based on the movement of the intestinal wall upon the suction, and changes the operation plan based on the result. For example, if the inspection status determination section 161 determines the presence of adhesion in the sigmoid colon, the inspection status determination section 161 determines that insertion using the usual shaft retention and shortening method is difficult. This is because the pulling operation in the shaft retention and shortening method may stimulate the adhesion and may cause pain. At this time, the support information generation section 150 presents a guide of the shaft retention and shortening method that takes the adhesion into account, or a guide of the Push method.

Further, when a polyp is removed at the time of treatment, the inspection status determination section 161 determines the size and the location of the polyp to thereby determine whether the polyp should be removed by suction or it should be removed by a treatment tool. The support information generation section 150 presents the determination result as the support information AST. If the inspection status determination section 161 determines that the polyp should be removed by suction, the control determination section 162 and the control section 170 adjust the suction amount according to the size or the condition of the polyp. Further, upon the suction of the polyp, water is made to automatically flow through the suction tube of the endoscope, making it easier for the polyp to flow to a point where it is caught.

### 7. Endoscope System and Fifth Detailed Configuration Example

The following describes a configuration example in which the control device 100 performs automatic suction control by further using other information than the endoscope image IMG.

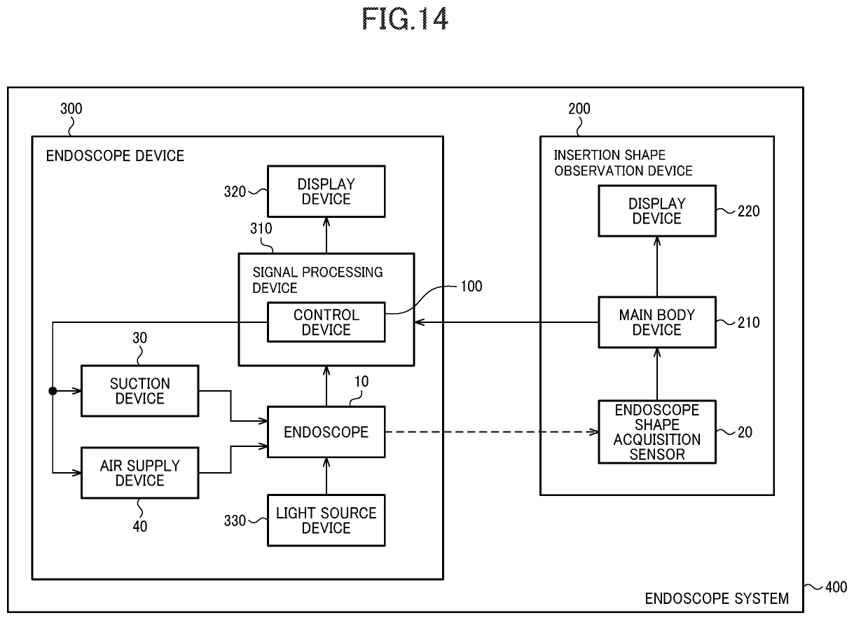

FIG. 14 illustrates a configuration example of an endoscope system 400 including the control device 100. The endoscope system 400 includes an endoscope device 300 and an insertion shape observation device 200. If the control device 100 does not use insertion shape information, the insertion shape observation device 200 may be omitted.

The endoscope device 300 includes an endoscope 10, a suction device 30, an air supply device 40, a light source device 330, a signal processing device 310, and a display device 320. The light source device 330 generates and controls the illumination light, which is guided by a light guide to the distal end of the endoscope 10, and emitted from the distal end of the endoscope 10. The signal processing device 310 generates endoscope images by processing the image signals output by the endoscope 10. The signal processing device 310 also acquires the ID or the like of the endoscope 10, as the endoscope type information. The display device 320 displays endoscope images generated by the signal processing device 310.

The insertion shape observation device 200 includes an endoscope shape acquisition sensor 20, a main body device 210, and a display device 220. The endoscope shape acquisition sensor 20 detects the magnetic field of the source coil provided in the endoscope insertion section. The main body device 210 acquires the position and the shape of the endoscope insertion section based on the detection signal from the endoscope shape acquisition sensor 20, and outputs the images showing the position and the shape of the endoscope insertion section to the display device 220. The display device 220 displays the images output by the main body device 210. The display device 220 and the display device 320 are also called a monitor and are liquid crystal display devices or the like. A single display device may be provided in the endoscope system 400, and the endoscope device 300 and the insertion shape observation device 200 may share the single display device.

The control device 100 is provided in the signal processing device 310. The endoscope images and endoscope type information from the signal processing device 310, as well as information such as the position, the shape, and the like, of the endoscope insertion section acquired by the main body device 210, are input to the control device 100. The control device 100 may be located anywhere within the endoscope system 400.

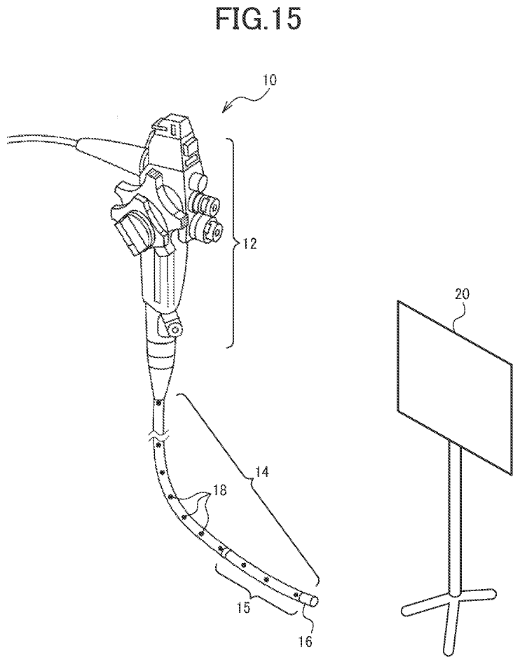

FIG. 15 illustrates configuration examples of the endoscope 10 and the endoscope shape acquisition sensor 20. As shown in FIG. 15, the endoscope 10 includes an operation section 12, an insertion section 14, and a source coil 18.

The insertion section 14 has a flexible, elongated shape and includes a rigid section 16 at its distal end and an angle-operable curving section 15. The rigid section 16 is equipped with an imaging device, an illumination lens, a water supply port, an air supply port, a forceps opening, and the like.

The operation section 12 is a device for allowing the user to operate the endoscope 10, and includes, for example, a grip section, an angle operating dial, an air/water supply button, and the like. The operation of the endoscope is described below. The user pushes the grip section in the longitudinal direction of the insertion section 14, so that the insertion section 14 is inserted. This action is called a pushing operation. Further, when the user pulls the grip section in the longitudinal direction of the insertion section 14, the insertion section 14 is pulled out. This action is called a pulling operation. By the user's action of turning the grip section in the circumferential direction of the insertion section 14, the insertion section 14 rotates in the circumferential direction. This action is called a torque operation. By the user's operation of the angle operating dial, the curving section 15 of the insertion section 14 bends up, down, left, and right. This action is called an angle operation.

The source coil 18 generates a magnetic field. For example, a plurality of source coils 18 are provided in the insertion section 14 at predetermined intervals. The endoscope shape acquisition sensor 20 detects the magnetic field from each source coil 18, and the main body device 210 of the insertion shape observation device 200 detects the position of each source coil 18 based on the detection signal, thereby detecting the position of each section of the insertion section 14. Further, the main body device 210 detects the shape of the insertion section 14 based on the detected positions of the plurality of source coils 18. The sensing method in the observation of the insertion shape is not limited to the method using magnetic fields, but may also be a method using electromagnetic waves, ultrasonic waves, lights, and the like, for example.

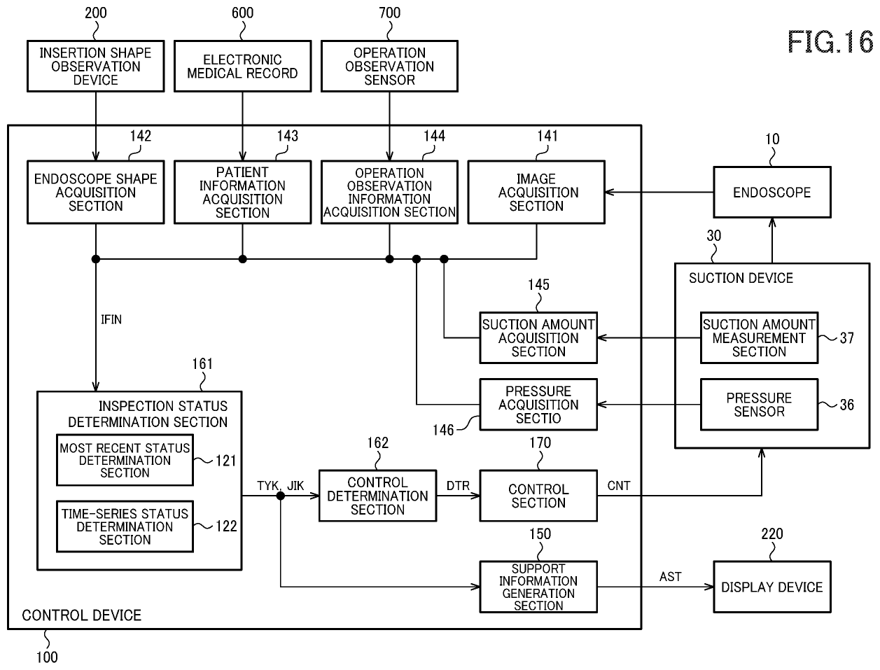

FIG. 16 illustrates a fifth detailed configuration example of the control device 100. In this configuration example, the control device 100 further includes an endoscope shape acquisition section 142, a patient information acquisition section 143, an operation observation information acquisition section 144, a suction amount acquisition section 145, and a pressure acquisition section 146. In this configuration example, the image acquisition section 141, the endoscope shape acquisition section 142, the patient information acquisition section 143, the operation observation information acquisition section 144, the suction amount acquisition section 145, and the pressure acquisition section 146 correspond to the information acquisition section 140 in FIG. 1. The same components as those already described will be omitted from the description as appropriate.

It is not necessary that all of the endoscope shape acquisition section 142, the patient information acquisition section 143, the operation observation information acquisition section 144, the suction amount acquisition section 145, and the pressure acquisition section 146 are provided. Any one to five of these sections may be omitted. Further, the support information generation section 150 may be omitted, or automatic air supply control may be further combined, as in the third detailed configuration example.

The endoscope shape acquisition section 142 acquires insertion shape information based on the detection signal from the endoscope shape acquisition sensor 20. The patient information acquisition section 143 acquires patient information from an electronic medical record 600. The operation observation information acquisition section 144 acquires operation observation information from an operation observation sensor 700. The image acquisition section 141 acquires endoscope images from the endoscope 10. The suction amount acquisition section 145 acquires measurement values of the suction amount from the suction amount measurement section 37. The pressure acquisition section 146 acquires measurement values of the pressure from the pressure sensor 36.

The insertion shape information, the patient information, the operation observation information, the endoscope image, the measurement value of suction amount, and the measurement value of pressure are input to the inspection status determination section 161 as the information IFIN. The inspection status determination section 161 determines the inspection status from among the plurality of inspection statuses based on the information IFIN. The inspection status includes, for example, an insertion status of the endoscope, an operation status, a state of the intestine, and the like. Specifically, the inspection status determination section 161 classifies the inspection status based on the information IFIN, and outputs the most recent status information TYK and the time-series status information JIK based on the classification result.

The insertion shape information is information of the position and the shape of the endoscope insertion section, and is acquired by the insertion section shape observation device described above. The inspection status determination section 161 may refer to the position and the shape of the endoscope insertion section to recognize their changes, and use the recognition result in the above classification.

By using the insertion shape information in the determination of the inspection status, the position and the shape of the insertion section or the changes thereof can be directly determined. For example, the site where the endoscope 10 is located, the operation being performed by the doctor, the amount of the operation, the procedures during the operation, the shape of the insertion section during the operation, and the like may be determined. This can improve the accuracy in the determination of inspection status.

The operation observation sensor 700 is a sensor capable of measuring the motion of a doctor, etc. who operates the endoscope 10, and is, for example, a motion capture sensor. The operation observation sensor 700 may be a sensor that is attached to the doctor, etc., or it may be a sensor that measures the motion of the doctor, etc. from a place distant from the doctor, etc. The operation observation information acquisition section 144 acquires the measured motion information of the doctor, etc. as the operation observation information.

By using the operation observation information in the determination of the inspection status, it is possible to directly determine the operation being performed by the doctor, etc. For example, it is possible to determine the operation being performed by the doctor, etc., the amount of the operation, the procedure during the operation, whether it is the correct procedure in the currently employed operation method, and the like. This can improve the accuracy in the determination of inspection status.

The electronic medical record 600 accumulates patient information, which is information about patient attributes. The electronic medical record 600 is stored in, for example, a storage device provided outside the endoscope system 400, and the patient information acquisition section 143 acquires the patient information from the storage device. The patient information is, for example, the constitution, gender, age, medical history, body fat percentage, CT image of the patient, or any combination of two or more of them. The constitution is BMI, height, weight, or any combination of two or more of them. Further, the patient information may also include past inspection information. The past inspection information refers to information as to which insertion method was used, which scope was used, history of suction upon the insertion, whether or not pain occurred, or any combination of two or more of these information items.

By using the patient information in the determination of the inspection status, presentation of support information or automatic suction control based on the information of the patient receiving the inspection can be achieved. For example, by using CT image, the shape of the intestine of the patient can be determined, and an ideal insertion plan can be created based on this information. At this time, the optimal automatic suction control for the insertion plan may be performed. In addition, an intestine MAP can be created and compared with each acquired information to sequentially present optimal guides. By superimposing the location of the polyp on the CT image at the time of observation or treatment in advance, the time required to search for each polyp can be reduced, thus allowing smooth transition to the treatment. It also enables determination as to whether the polyp found has been previously present or it is a polyp newly found.

The pressure sensor 36 is as described above in FIG. 10. By using the measurement value of the pressure in the determination of the inspection status, the luminal status can be determined based on the internal pressure of lumen. This can improve the accuracy in the determination of inspection status. For example, excessive air supply or the like can be determined from the internal pressure of lumen. Also, when the suction fails due to blockage of suction tube or the like, the failure can be presumed from the measurement value of the pressure sensor. Therefore, when the intestinal wall does not come closer by the suction, it is possible to determine whether it is due to adhesion or the like or malfunction.

The suction amount measurement section 37 is as described above in FIG. 10. By using the measurement value of the suction amount in the determination of the inspection status, the inspection status can be determined based on the suction amount. This can improve the accuracy in the determination of inspection status. For example, it is possible to determine whether the suction is turned on or off at an appropriate timing, whether the suction is performed with an appropriate suction strength, or the like.

Although the embodiments to which the present disclosure is applied and the modifications thereof have been described in detail above, the present disclosure is not limited to the embodiments and the modifications thereof, and various modifications and variations in components may be made in implementation without departing from the spirit and scope of the present disclosure. The plurality of elements disclosed in the embodiments and the modifications described above may be combined as appropriate to implement the present disclosure in various ways. For example, some of all the elements described in the embodiments and the modifications may be deleted. Furthermore, elements in different embodiments and modifications may be combined as appropriate. Thus, various modifications and applications can be made without departing from the spirit and scope of the present disclosure. Any term cited with a different term having a broader meaning or the same meaning at least once in the specification and the drawings can be replaced by the different term in any place in the specification and the drawings.

1. A control device comprising:
a processor, wherein
the processor is configured to:
acquire an endoscope image that is an image captured by an endoscope;
determine whether suction is needed, based on the endoscope image;
when the suction is determined to be needed, implement control to carry out the suction;
determine a state of a subject based on a change in the endoscope image when the suction is performed; and
perform control regarding suction based on the state of the subject.

2. The control device as defined in claim 1, wherein
the processor determines whether the suction is needed according to a distance between a mucosa of the subject and a distal end section of the endoscope, based on the endoscope image.

3. The control device as defined in claim 2, wherein
the processor
determines whether the distance between the mucosa of the subject and the distal end section of the endoscope is longer than a predetermined distance, based on the endoscope image, and,
when the distance is determined to be longer than the predetermined distance, determines that the suction is needed.

4. The control device as defined in claim 3, wherein
the predetermined distance is an optimal distance with which folds of a large intestine can be flipped by an angle operation and a torque operation of the endoscope in a shaft retention and shortening method.

5. The control device as defined in claim 1, wherein
the processor determines whether the suction is needed based on a change in the endoscope image when the suction is performed.

6. The control device as defined in claim 5, wherein
the processor determines not to perform the suction when a distance between a mucosa of the subject and a distal end section of the endoscope is not shorter than a predetermined distance, based on a change in the endoscope image when the suction is performed.

7. The control device as defined in claim 5, wherein
the processor determines not to perform the suction when presence of adhesion is detected as a result of determination of presence or absence of the adhesion in the subject, based on a change in the endoscope image when the suction is performed.

8. The control device as defined in claim 5, wherein
the processor determines an operation plan indicating an operation procedure of the endoscope based on a change in the endoscope image when the suction is performed, and determines whether the suction is needed according to the determined operation plan.

9. The control device as defined in claim 1, wherein
the processor determines whether the suction is needed according to a position of the endoscope in a lumen based on the endoscope image.

10. The control device as defined in claim 9, wherein
the processor determines that the suction is needed when the endoscope is determined to be located in a predetermined site in the lumen based on the endoscope image.

11. The control device as defined in claim 10, wherein
the predetermined site is a portion beyond a mid-trans in a sigmoid colon or a transverse colon.

12. The control device as defined in claim 1, wherein
the processor determines not to perform the suction when a red ball state, in which a distal end section of the endoscope is in contact with a mucosa of the subject, is detected based on the endoscope image.

13. The control device as defined in claim 1, wherein
the processor determines to perform the suction when an excessive air supply state, in which a lumen is swelled more than a predetermined level as a result of air supply, is detected based on the endoscope image.

14. The control device as defined in claim 1, wherein
the processor
determines a strength of the suction based on the endoscope image; and
implements control to carry out the suction with the determined strength.

15. The control device as defined in claim 14, wherein
the processor determines the strength of the suction according to a distance between a mucosa of the subject and a distal end section of the endoscope, based on the endoscope image.

16. The control device as defined in claim 15, wherein
the processor weakens the suction as the distance between the mucosa of the subject and the distal end section of the endoscope becomes closer by the suction.

17. The control device as defined in claim 14, wherein
the processor recognizes at least one of a residue, water, a resected tissue, or a sprayed chemical liquid based on the endoscope image, and determines whether the suction is needed and determines the strength of the suction, according to the recognition result.

18. The control device as defined in claim 1, wherein
the processor acquires insertion shape information, which is information representing a shape of an insertion section of the endoscope; and
determines whether the suction is needed based on the endoscope image and the insertion shape information.

19. The control device as defined in claim 1, wherein
the processor
acquires inspection status information, which is information regarding a status of endoscopy using the endoscope, based on the endoscope image, and
determines whether the suction is needed based on the inspection status information.

20. A control method, comprising:
acquiring an endoscope image that is an image captured by an endoscope;
determining whether suction is needed, based on the endoscope image;
when the suction is determined to be needed, implementing control to carry out the suction;
determining a state of a subject based on a change in the endoscope image when the suction is performed; and
performing control regarding suction based on the state of the subject.
